SEQUENCE LISTING (26 sequences)

SEQ ID NO: 1 - DNA, 20 bp, synthetic construct
  source 1..20: mol_type = other DNA
agagtttgatcmtggctcag

SEQ ID NO: 2 - DNA, 22 bp, synthetic construct
  source 1..22: mol_type = other DNA
tacggytaccttgttacgactt

SEQ ID NO: 3 - DNA, 1198 bp, unidentified
  source 1..1198: mol_type = other DNA
agctagggtgggtacggattattgggtttaagggtccgtaggcggatgtgtaagtcagtggtgaaatctcacagcttaactgtgaaactgccattgatactgcatgtcttgagtaaggtagaagtggctggaataagtagtgtagcggtgaaatgcatagatattacttagaacaccaattgcgaaggcaggtcactatgtcttaactgacgctgatggacgaaagcgtggggagcgaacaggattagataccctggtagtccacgccgtaaacgatgctaactcgtttttgggttttcggattcagagactaagcgaaagtgataagttagccacctggggagtacgttcgcaagaatgaaactcaaaggaattgacgggggcccgcacaagcggtggattatgtggtttaattcgatgatacgcgaggaaccttaccaaggcttaaatgggaattgatcggtttagaaatagaccttccttcgggcaattttcaaggtgctgcatggttgtcgtcagctcgtgccgtgaggtgttaggttaagtcctgcaacgagcgcaacccctgttactagttgctaccattaagttgaggactctagtaagactgcctacgcaagtagagaggaaggtggggatgacgtcaaatcatcacggcccttacgccttgggccacacacgtaatacaatggcaggtacagagggcagctacacagcgatgtgatgcgaatctcgaaagcctgtctcagttcggattggagtctgcaactcgactctatgaagctggaatcgctagtaatcgcgcatcagccatggcgcggtgaatacgttcccgggccttgtacacaccgcccgtcaagccatggaagtctggggtacctgaagtcggtgaccgtaaaaggagctgcctagggtaaaacaggtaactagggctaatcgtacaggggaaaccctaaaagtttaaccaggaaccggtagaaggtgttaaggatagagtgtatatctgttttgtatatgttgtatttatctagtgtatctgagataatggatagataacgctcgaactggcactacactagtgagagatattagtattcttatatattaagtctctattgagatatcataatcgtggataaagacttgacagcggcgagagcctggatagcggagtggtgttagctatgagtgtataatc

SEQ ID NO: 4 - DNA, 42 bp, synthetic construct
  source 1..42: mol_type = other DNA
gaagaaggagatatacatatgcaggaccgcaatgcagcactt

SEQ ID NO: 5 - DNA, 47 bp, synthetic construct
  source 1..47: mol_type = other DNA
gtggtggtggtggtggtgctcgagctctttaataacttttttggaaa

SEQ ID NO: 6 - DNA, 41 bp, synthetic construct
  source 1..41: mol_type = other DNA
gctgcggccggtgcacatatgcagacttttatccaggcata

SEQ ID NO: 7 - DNA, 44 bp, synthetic construct
  source 1..44: mol_type = other DNA
gtggtgatgatggtgatgtctagaccttcctaccaggatctttc

SEQ ID NO: 8 - DNA, 41 bp, synthetic construct
  source 1..41: mol_type = other DNA
gctgcggccggtgcacatatgcaacagctgagtgcagaaca

SEQ ID NO: 9 - DNA, 44 bp, synthetic construct
  source 1..44: mol_type = other DNA
gtggtgatgatggtgatgtctagagtatttgataaattttatat

SEQ ID NO: 10 - DNA, 41 bp, synthetic construct
  source 1..41: mol_type = other DNA
gaagaaggagatatacatatgtgcaacaccaatgaaatgag

SEQ ID NO: 11 - DNA, 44 bp, synthetic construct
  source 1..44: mol_type = other DNA
gtggtggtggtggtggtgctcgagtggggcatccgggctgatcc

SEQ ID NO: 12 - DNA, 41 bp, synthetic construct
  source 1..41: mol_type = other DNA
gaagaaggagatatacatatgaacaatctgctacaacagcc

SEQ ID NO: 13 - DNA, 44 bp, synthetic construct
  source 1..44: mol_type = other DNA
gtggtggtggtggtggtgctcgagtttcagctgcttaaattctt

SEQ ID NO: 14 - DNA, 42 bp, synthetic construct
  source 1..42: mol_type = other DNA
gctgcggccggtgcacatatggacaacattgcaaaccaacac

SEQ ID NO: 15 - DNA, 45 bp, synthetic construct
  source 1..45: mol_type = other DNA
gtggtgatgatggtgatgtctagagtacaatctcaatcctgatct

SEQ ID NO: 16 - DNA, 41 bp, synthetic construct
  source 1..41: mol_type = other DNA
gctgcggccggtgcacatatgcaaaaaaacaacgatgaaat

SEQ ID NO: 17 - DNA, 44 bp, synthetic construct
  source 1..44: mol_type = other DNA
gtggtgatgatggtgatgtctagattctttgatcacctttttgg

SEQ ID NO: 18 - DNA, 41 bp, synthetic construct
  source 1..41: mol_type = other DNA
gctgcggccggtgcacatatgcagctggtgtttgtgtattt

SEQ ID NO: 19 - DNA, 44 bp, synthetic construct
  source 1..44: mol_type = other DNA
gtggtgatgatggtgatgtctagattcctttatgaatttttcat

SEQ ID NO: 20 - protein, 275 aa, unidentified
CNDNSTEDNNLLQQPQAESAKVEQPNEREKACYYVDQNWNSSAVLKTTLKTSTDTNFMNGQMTKIASLWGRNNPTLRFVDDPSSPNSTYNAISYSTGKIYYGYAIYYDAKNKGGDIVNAMILAHEYGHQLQYIFGLPSVNESTARPNELEADGFAGYYLRRPEGYNKTQFSEIAAAYEFAQSIGDYATTSPGHHGTPPQRRSAVRLGFLLGQYNLSASDFDYNFFYYYQGVLNGTYKMGKNSRNPELDAYMMQYIDELRKIQSGEISAEEFKQLK

SEQ ID NO: 21 - protein, 21 aa, unidentified
MKKNFNFCCLAGAIAVFSLTA

SEQ ID NO: 22 - protein, 296 aa, unidentified
MKKNFNFCCLAGAIAVFSLTACNDNSTEDNNLLQQPQAESAKVEQPNEREKACYYVDQNWNSSAVLKTTLKTSTDTNFMNGQMTKIASLWGRNNPTLRFVDDPSSPNSTYNAISYSTGKIYYGYAIYYDAKNKGGDIVNAMILAHEYGHQLQYIFGLPSVNESTARPNELEADGFAGYYLRRPEGYNKTQFSEIAAAYEFAQSIGDYATTSPGHHGTPPQRRSAVRLGFLLGQYNLSASDFDYNFFYYYQGVLNGTYKMGKNSRNPELDAYMMQYIDELRKIQSGEISAEEFKQLK

SEQ ID NO: 23 - protein, 19 aa, unidentified
MKKLLLGALMLGFMSACNS

SEQ ID NO: 24 - protein, 331 aa, unidentified
MKKLLLGALMLGFMSACNSDNIANQHEQSDNLVSPTGGSALQRGCASEEVRKIALQNSPELRQRFSALEAQTEKFENDLKLGKVLSDGSVEIPVVVNVLYRTSSENLSDSRIAEQIAVLNADYAGTNSDVSKIPSEFQSVKSGDVKVKFRLANTVRKSTTKTSWSTNDAMKRSSSGGIDATSPSNYLNIWVVGNMGQILGYATFPESAGMWNDGVVIAAPYFGKTGASSPFNLGRTATHEVGHYLNLRHIWGDANCGNDLVADTPTQTGANSGKPNYPLYNTCSGVQRSVMFMNYMDYVDDAAMFMFSAGQRTRMQSVVASSGPRSGLRLY

SEQ ID NO: 25 - protein, 19 aa, unidentified
MKTMLKSILTVALLWSAVS

SEQ ID NO: 26 - protein, 281 aa, unidentified
MKTMLKSILTVALLWSAVSCNTNEMSDINSSAQTQTSNSGNFAAKASDNPENPLNNWEECGKKHNVILTYVITYQKTLDNSGLTNSEIADKCIMASNNYFNQKYAQDYNNTQSPFTVAKIRSILNDGDNKFSNVIQGMNASDKVKAKTSELVNMMYNLASTDKNTEYAEVKARIVDFEAGIMNDASLSQAEKDQLLQTTSIARYSSYLWSNTIDHSDSVSSVTGKKKWWKWVVVAVCDFAGGAAGTAAGGGVLSVAGAIAGAAGASSGGAALVDWISPDAP

(19) United States
(12) Patent Application Publication
(10) Pub. No.: US 2026/0115120 A1
(43) Pub. Date: Apr. 30, 2026

(54) COMPOSITION FOR INHIBITING LIPOFUSCIN ACCUMULATION OR REMOVING LIPOFUSCIN CONTAINING METALLOPROTEASE DERIVED FROM A STRAIN OF THE GENUS CHRYSEOBACTERIUM

(71) Applicant: AMOREPACIFIC CORPORATION, Seoul (KR)

(72) Inventors: Eunmi KIM, Yongin-si (KR); Si Young CHO, Yongin-si (KR)

(73) Assignee: AMOREPACIFIC CORPORATION, Seoul (KR)

(21) Appl. No.: 19/364,172

(22) Filed: Oct. 21, 2025

(30) Foreign Application Priority Data
Oct. 29, 2024 (KR) 10-2024-0149635

(51) Int. Cl. A61K 8/66; A61K 8/99; A61K 35/74; A61K 38/48; A61Q 19/02; A61Q 19/08; C12R 1/01

(52) U.S. Cl. CPC A61K 8/66; A61K 8/99; A61K 35/74; A61K 38/4886; A61Q 19/02; A61Q 19/08; C12R 2001/01; C12Y 304/24004

(57) ABSTRACT

The present disclosure discloses a composition for skin anti-aging, skin lightening, or skin texture improvement containing a metalloproteinase derived from a strain of the genus Chryseobacterium as an active ingredient, and/or a composition for inhibition of lipofuscin accumulation or for removal of lipofuscin containing the metalloproteinase as an active ingredient. The composition provides an efficacy of skin anti-aging, skin lightening, or skin texture improvement by inhibition of lipofuscin accumulation or by removal of lipofuscin, and provides an effect of preventing, improving, or treating a disease caused by lipofuscin accumulation.

### CROSS REFERENCE TO RELATED APPLICATION

The present application claims priority to Korean Patent Application No. 10-2024-0149635, filed Oct. 29, 2024, the entire contents of which are hereby incorporated by this reference.

### INCORPORATION BY REFERENCE OF SEQUENCE LISTING

The instant application contains a Sequence Listing which has been filed electronically in xml format and is hereby incorporated by reference in its entirety. Said xml copy, created on Oct. 21, 2025, is named Q314515SequenceListing.xml and is 26,642 bytes in size.

### BACKGROUND OF THE INVENTION

### Field of the Invention

The present disclosure discloses a composition for skin anti-aging, skin lightening, skin texture improvement, inhibition of lipofuscin accumulation, and/or removal of lipofuscin, comprising, as an active ingredient, a metalloproteinase derived from a strain of the genus Chryseobacterium.

### Description of the Related Art

Lipofuscin is a metabolic product related to aging or oxidative stress, and exists as a form of insoluble pigment within cells. Lipofuscin is known to be formed as incomplete degradation substances accumulate within cells due to oxidative stress and decline of cellular performance. As lipofuscin accumulates within cells, cellular function decreases and the aging rate accelerates.

The heart of long-lived elderly becomes brown and smaller, which is because lipofuscin pigment increases, and this is called brown atrophy of heart. Such pigmentation may also occur in liver, and this is called brown atrophy of liver. In addition, cases of lipofuscin pigmentation in heart, liver, and kidney have been reported in patients with cancer or patients who have suffered for a long time from wasting diseases such as severe pulmonary tuberculosis. Meanwhile, when skin ages, the most prominent phenomenon is pigmentation such as melasma and liver spots, and such pigmentation is a phenomenon appearing by accumulation of lipofuscin. Accordingly, lipofuscin is also called “liver spot of the body.” Therefore, inhibiting generation and/or accumulation of lipofuscin so that lipofuscin does not accumulate in skin cells is very important in preventing skin aging.

As described above, lipofuscin that naturally occurs in cellular metabolic processes accumulates in larger amounts within cells not only in aging but also when cellular function decreases or when repeatedly exposed to external toxins. In addition, accumulated lipofuscin interferes with normal function of cells and further promotes the aging rate. Lipofuscin is found not only in skin cells but also in liver, kidney, cardiac muscle, retina, adrenal gland, nerve cells and ganglion cells, and is known to cause various degenerative diseases by accumulating in important body organs. For example, when lipofuscin accumulates in cardiac muscle, it may deteriorate cardiac muscle and induce myocardial infarction, and when lipofuscin accumulates in retina or nerve cells, it may induce macular degeneration or Alzheimer's disease. In addition, when lipofuscin accumulates in organs such as adrenal gland, liver, pituitary gland, and the like that secrete hormones, abnormalities may occur in metabolic functions of body hormones such as growth hormone, sex hormone, and the like.

Observation of lipofuscin in skin has been regarded as a kind of aging sign, and research regarding cosmetic materials that help to reduce this has been conducted. However, until now, there has been no cosmetic material known to directly degrade lipofuscin, and only indirect effects have been expected through some ingredients that reduce oxidative stress and promote regeneration of skin cells.

### SUMMARY OF THE INVENTION

In one aspect, the present disclosure is directed to providing a composition for skin anti-aging, skin lightening, or skin texture improvement, comprising, as an active ingredient, a metalloproteinase derived from a strain of the genus Chryseobacterium.

In another aspect, the present disclosure is directed to providing a composition for inhibition of lipofuscin accumulation or removal of lipofuscin, comprising, as an active ingredient, a metalloproteinase derived from a strain of the genus Chryseobacterium.

In one aspect, the present disclosure provides a composition for skin anti-aging, skin lightening, or skin texture improvement, comprising, as an active ingredient, a metalloproteinase derived from a strain of the genus Chryseobacterium.

In an exemplary embodiment, the strain may be a strain of Chryseobacterium camelliae.

In an exemplary embodiment, the strain may be a Chryseobacterium camelliae Dolsongi-HT1 strain with a deposit number of KCCM11883P.

In an exemplary embodiment, the metalloproteinase may comprise an amino acid sequence of SEQ ID NO: 20 or SEQ ID NO: 22.

In an exemplary embodiment, the composition may provide efficacy of skin anti-aging, skin lightening, or skin texture improvement by inhibition of lipofuscin accumulation or removal of lipofuscin.

In another aspect, the present disclosure provides a composition for inhibition of lipofuscin accumulation or removal of lipofuscin, comprising as an active ingredient a metalloproteinase derived from a strain of the genus Chryseobacterium.

In an exemplary embodiment, the strain may be a strain of Chryseobacterium camelliae.

In an exemplary embodiment, the strain may be a Chryseobacterium camelliae Dolsongi-HT1 strain with a deposit number of KCCM11883P.

In an exemplary embodiment, the metalloproteinase may comprise an amino acid sequence of SEQ ID NO: 20 or SEQ ID NO: 22.

In an exemplary embodiment, the composition may prevent, improve, or treat a disease caused by lipofuscin accumulation.

In an exemplary embodiment, the disease caused by lipofuscin accumulation may comprise one or more selected from the group consisting of sarcopenia, progeria, Alzheimer's disease, Parkinson's disease, amyotrophic lateral sclerosis (ALS), myocardial infarction, age-related macular degeneration, neuronal ceroid lipofuscinoses (NCL), acromegaly, denervation atrophy, and chronic obstructive pulmonary disease (COPD).

In one aspect, the technology disclosed in the present disclosure has an effect of providing a material capable of degrading lipofuscin. The metalloproteinase derived from a strain of the genus Chryseobacterium provides an effect of directly degrading lipofuscin accumulated within cells.

In another aspect, the technology disclosed in the present disclosure has an effect of providing a composition for skin anti-aging, skin lightening, or skin texture improvement, comprising, as an active ingredient, a metalloproteinase derived from a strain of the genus Chryseobacterium.

In still another aspect, the technology disclosed in the present disclosure has an effect of providing a composition for inhibition of lipofuscin accumulation or removal of lipofuscin, comprising, as an active ingredient, a metalloproteinase derived from a strain of the genus Chryseobacterium.

### BRIEF DESCRIPTION OF THE DRAWINGS

FIG. 1 illustrates a partial sequence of 16S rDNA of a Chryseobacterium camelliae Dolsongi-HT1 strain (SEQ ID NO: 3) according to one embodiment.

FIG. 2 illustrates results of evaluation of keratin degradation activity according to one embodiment.

FIG. 3 illustrates results of evaluation of lipofuscin removal activity according to one embodiment.

### DETAILED DESCRIPTION OF THE INVENTION

Hereinafter, the present disclosure will be described in detail.

In the present disclosure, “active ingredient” means an ingredient that may exhibit the intended activity alone or exhibit the intended activity in combination with a carrier itself having no activity and the like.

In one aspect, the present disclosure provides a composition for skin anti-aging, skin lightening, and/or skin texture improvement, comprising, as an active ingredient, a metalloproteinase derived from a strain of the genus Chryseobacterium.

In another aspect, the present disclosure provides a method for skin anti-aging, skin lightening, and/or skin texture improvement, comprising applying or administering to a subject in need thereof a composition comprising an effective amount of a metalloproteinase derived from a strain of the genus Chryseobacterium for skin anti-aging, skin lightening, and/or skin texture improvement.

In another aspect, the present disclosure provides a composition comprising a metalloproteinase derived from a strain of the genus Chryseobacterium for use in skin anti-aging, skin lightening, and/or skin texture improvement.

In another aspect, the present disclosure provides a use of a composition comprising a metalloproteinase derived from a strain of the genus Chryseobacterium for skin anti-aging, skin lightening, and/or skin texture improvement.

In another aspect, the present disclosure provides a use of a composition comprising a metalloproteinase derived from a strain of the genus Chryseobacterium in manufacturing a composition for skin anti-aging, skin lightening, and/or skin texture improvement.

In another aspect, the present disclosure provides a use of a composition comprising a metalloproteinase derived from a strain of the genus Chryseobacterium in manufacturing a composition for melasma improvement.

In another aspect, the present disclosure provides a use of a composition comprising a metalloproteinase derived from a strain of the genus Chryseobacterium in manufacturing a food composition for melasma improvement.

In an exemplary embodiment, the composition or the metalloproteinase may provide efficacy of skin anti-aging, skin lightening, and/or skin texture improvement by inhibition of lipofuscin accumulation or removal of lipofuscin.

In an exemplary embodiment, the skin anti-aging, skin lightening, and/or skin texture improvement may be obtained by inhibition of lipofuscin accumulation or removal of lipofuscin.

In an exemplary embodiment, the melasma improvement may be obtained by inhibition of lipofuscin accumulation or removal of lipofuscin.

In the present disclosure, “anti-aging” means a use of preventing, delaying, and/or improving an aging phenomenon occurring by internal factors including genetic factors and the like and external factors including ultraviolet rays and the like. The composition may provide an effect of preventing, improving, and/or treating a disease or symptom related to aging of cells by decreasing a level of aging of cells. For example, the anti-aging may be preventing, delaying, and/or improving aging by accumulation of lipofuscin.

In an exemplary embodiment, the composition or the metalloproteinase may prevent, improve, and/or treat liver spot and/or melasma.

In an exemplary embodiment, the composition or the metalloproteinase may make skin clear, bright, or transparent by inhibiting lipofuscin accumulation or removing lipofuscin, and may help in whitening and/or skin tone improvement.

In another aspect, the present disclosure provides a composition for inhibition of lipofuscin accumulation or removal of lipofuscin, comprising, as an active ingredient, a metalloproteinase derived from a strain of the genus Chryseobacterium.

In another aspect, the present disclosure provides a method for inhibition of lipofuscin accumulation or removal of lipofuscin, comprising applying or administering to a subject in need thereof a composition comprising an effective amount of a metalloproteinase derived from a strain of the genus Chryseobacterium for inhibiting lipofuscin accumulation or removing lipofuscin.

In an exemplary embodiment, the method may improve or treat a disease caused by lipofuscin accumulation.

In an exemplary embodiment, the method may have an anti-aging effect on a subject.

In an exemplary embodiment, the method may brighten skin color of a subject.

In an exemplary embodiment, the method may improve skin texture of a subject.

In another aspect, the present disclosure provides a composition comprising a metalloproteinase derived from a strain of the genus Chryseobacterium for use in inhibition of lipofuscin accumulation or removal of lipofuscin.

In another aspect, the present disclosure provides a use of a composition comprising a metalloproteinase derived from a strain of the genus Chryseobacterium for inhibition of lipofuscin accumulation or removal of lipofuscin.

In another aspect, the present disclosure provides a use of a composition comprising a metalloproteinase derived from a strain of the genus Chryseobacterium in manufacturing a composition for inhibition of lipofuscin accumulation or removal of lipofuscin.

In an exemplary embodiment, the composition or the metalloproteinase may prevent, improve, and/or treat a disease caused by lipofuscin accumulation.

In an exemplary embodiment, the composition or the metalloproteinase may have an anti-aging effect on a subject.

In an exemplary embodiment, the composition or the metalloproteinase may brighten skin color of a subject.

In an exemplary embodiment, the composition or the metalloproteinase may improve skin texture of a subject.

In an exemplary embodiment, the disease caused by lipofuscin accumulation may comprise one or more selected from the group consisting of sarcopenia, progeria, Alzheimer's disease, Parkinson's disease, amyotrophic lateral sclerosis (ALS), myocardial infarction, age-related macular degeneration, neuronal ceroid lipofuscinoses (NCL), acromegaly, denervation atrophy, and chronic obstructive pulmonary disease (COPD).

In an exemplary embodiment, the strain may be a strain of Chryseobacterium camelliae.

In an exemplary embodiment, the strain may be a Chryseobacterium camelliae Dolsongi-HT1 strain with a deposit number of KCCM11883P.

In an exemplary embodiment, the strain may be isolated from green tea leaves.

In an exemplary embodiment, the Chryseobacterium camelliae Dolsongi-HT1 strain may have a partial sequence of 16S rDNA of SEQ ID NO: 3.

In an exemplary embodiment, the metalloproteinase may comprise an amino acid sequence of SEQ ID NO: 20 or SEQ ID NO: 22.

In an exemplary embodiment, the metalloproteinase may consist of an amino acid sequence of SEQ ID NO: 20 or SEQ ID NO: 22.

A peptide required to export a peptide or protein produced in a cell to the outside of the cell is called a signal peptide or a signal sequence. These play a role in guiding a protein produced in a cell toward the cell wall side and ultimately helping it to be secreted through the cell membrane. In this case, as the protein is secreted to the outside of the cell, the signal peptide is cut off, and the remaining parts form a structure and perform a function.

The metalloproteinase according to the present disclosure may be represented by an amino acid sequence of SEQ ID NO: 22 including a signal sequence of SEQ ID NO: 21.

SEQ ID NO: 21
MKKNFNFCCLAGAIAVFSLTA
SEQ ID NO: 22
MKKNFNFCCLAGAIAVFSLTACNDNSTEDNNLLQQPQAESAKVEQPNER
EKACYYVDQNWNSSAVLKTTLKTSTDTNFMNGQMTKIASLWGRNNPTLR
FVDDPSSPNSTYNAISYSTGKIYYGYAIYYDAKNKGGDIVNAMILAHEY
GHQLQYIFGLPSVNESTARPNELEADGFAGYYLRRPEGYNKTQFSEIAA
AYEFAQSIGDYATTSPGHHGTPPQRRSAVRLGFLLGQYNLSASDFDYNF
FYYYQGVLNGTYKMGKNSRNPELDAYMMQYIDELRKIQSGEISAEEFKQ
LK

In an exemplary embodiment, the composition comprising a metalloproteinase derived from a strain of the genus Chryseobacterium may be a cosmetic composition, an oral composition, a non-therapeutic oral composition, or a pharmaceutical composition.

In an exemplary embodiment, the composition comprising a metalloproteinase derived from a strain of the genus Chryseobacterium may be applied, administered, or delivered to a subject in the form of a cosmetic composition, an oral composition, a non-therapeutic oral composition, or a pharmaceutical composition.

According to an exemplary embodiment, the composition comprising a metalloproteinase derived from a strain of the genus Chryseobacterium may be a cosmetic composition.

The cosmetic composition may further include functional additives and ingredients included in general cosmetic compositions. The functional additives may include ingredients selected from the group consisting of water-soluble vitamins, oil-soluble vitamins, polymer peptides, polymer polysaccharides, sphingolipids, and seaweed extracts. Other blended ingredients included in the cosmetic composition, there may be oil ingredients, moisturizers, emollients, surfactants, organic and inorganic pigments, organic powders, ultraviolet absorbers, preservatives, bactericides, antioxidants, plant extracts, pH adjusters, alcohols, dyes, fragrances, blood circulation promoters, cooling agents, antiperspirants, purified water, and the like.

The cosmetic composition is not particularly limited in formulation and may be appropriately selected according to intended purpose. For example, the cosmetic composition may be manufactured in one or more formulations selected from the group consisting of skin lotion, skin softener, skin toner, astringent, lotion, milk lotion, moisture lotion, nourishing lotion, massage cream, nourishing cream, moisture cream, hand cream, foundation, essence, nourishing essence, pack, soap, cleansing foam, cleansing lotion, cleansing cream, body lotion, and body cleanser, but is not limited thereto.

When the formulation of the composition is paste, cream, or gel, as a carrier ingredient there may be used animal fibers, vegetable fibers, waxes, paraffins, starches, tragacanth, cellulose derivatives, polyethylene glycol, silicone, bentonite, silica, talc, zinc oxide, or the like.

When the formulation of the composition is powder or spray, as a carrier ingredient there may be used lactose, talc, silica, aluminum hydroxide, calcium silicate, or polyamide powder, and particularly in case of spray, a propellant such as chlorofluorohydrocarbon, propane/butane, or dimethyl ether may be further included.

When the formulation of the composition is solution or emulsion, as a carrier ingredient there may be used a solvent, solvating agent, or emulsifying agent, for example, water, ethanol, isopropanol, ethyl carbonate, ethyl acetate, benzyl alcohol, benzyl benzoate, propylene glycol, 1,3-butyl glycol oil, glycerol aliphatic ester, polyethylene glycol, or fatty acid ester of sorbitan.

When the formulation of the composition is suspension, as a carrier ingredient there may be used liquid diluents such as water, ethanol, or propylene glycol, suspending agents such as ethoxylated isostearyl alcohol, polyoxyethylene sorbitol ester, and polyoxyethylene sorbitan ester, microcrystalline cellulose, aluminum metahydroxide, bentonite, agar, tragacanth, or the like.

When the formulation of the composition is surfactant-containing cleansing, as a carrier ingredient there may be used aliphatic alcohol sulfates, aliphatic alcohol ether sulfates, sulfosuccinate monoesters, isethionates, imidazolinium derivatives, methyl taurates, sarcosinates, fatty acid amide ether sulfates, alkylamido betaines, aliphatic alcohols, fatty acid glycerides, fatty acid diethanolamides, vegetable oils, lanolin derivatives, ethoxylated glycerol fatty acid esters, or the like.

According to an exemplary embodiment, the composition comprising a metalloproteinase derived from a strain of the genus Chryseobacterium may be an oral composition or a non-therapeutic oral composition.

The oral composition or the non-therapeutic oral composition may be a formulation of liquid or solid state, and ingredients conventionally used in the relevant field may be appropriately selected and added by a person skilled in the art depending on formulation or intended use without difficulty, and a synergistic effect may occur when applied together with other raw materials.

The liquid formulation may include, as additional ingredients, various flavoring agents, natural carbohydrates, etc. like conventional beverages. Examples of the natural carbohydrates include common sugars, for example, monosaccharides such as glucose, fructose, and the like, disaccharides such as maltose, sucrose, and the like, polysaccharides such as dextrin, cyclodextrin, and the like, and sugar alcohols such as xylitol, sorbitol, erythritol, and the like. As the flavoring agents, natural flavoring agents (thaumatin, stevia extract (for example, rebaudioside A, glycyrrhizin, etc.)) and synthetic flavoring agents (for example, saccharin, aspartame, etc.) may be advantageously used. The ratio of the natural carbohydrates may generally be about 1 to 20 g per 100 ml of the composition, and in one aspect, may be about 5 to 12 g.

The oral composition or the non-therapeutic oral composition may, in one aspect, include various nutrients, vitamins, minerals (electrolytes), flavoring agents such as synthetic flavoring agents, natural flavoring agents, and the like, coloring agents and extending agents (cheese, chocolate, etc.), pectic acid and salts thereof, alginic acid and salts thereof, organic acids, protective colloid thickeners, pH adjusters, stabilizers, preservatives, glycerin, alcohols, carbonating agents used in carbonated beverages, and the like. In another aspect, the oral composition or the non-therapeutic oral composition may include fruit pulps for manufacture of natural fruit juices and vegetable beverages. The ingredients may be used independently or in combination. The ratio of the additives may vary, but generally is selected in a range of 0.001 to about 20 parts by weight per 100 parts by weight of the composition.

According to an exemplary embodiment, the composition comprising a metalloproteinase derived from a strain of the genus Chryseobacterium may be a pharmaceutical composition.

The pharmaceutical composition may further contain pharmaceutical adjuvants such as preservatives, stabilizers, hydrating agents or emulsification promoters, salts and/or buffers for osmotic pressure control, and the like, and other therapeutically useful substances, and may be formulated into various forms of oral administration agents or non-oral administration agents according to conventional methods.

The formulation for the oral administration agents may be, for example, tablets, pills, hard and soft capsules, liquids, suspensions, emulsions, syrups, powders, unit-dose powders, fine granules, pellets, and the like, and these formulations may contain surfactants, diluents (for example, lactose, dextrose, sucrose, mannitol, sorbitol, cellulose, and glycine), and lubricants (for example, silica, talc, stearic acid and magnesium or calcium salts thereof, and polyethylene glycol). The tablets may contain binders such as magnesium aluminum silicate, starch paste, gelatin, tragacanth, methyl cellulose, sodium carboxymethyl cellulose, and polyvinylpyrrolidone, and may contain pharmaceutical additives such as disintegrants like starch, agar, alginic acid or sodium salts thereof, absorbents, coloring agents, flavoring agents, sweetening agents, and the like, as occasion demands. The tablets may be manufactured by conventional mixing, granulating, or coating methods.

In addition, the non-oral administration forms may be transdermal administration formulations, and for example, may be formulations such as injections, drips, ointments, lotions, gels, creams, sprays, suspensions, emulsions, patches, or the like, but are not limited thereto.

Determination of an administration amount of the active ingredient is within the level of an ordinary skilled person, and a daily administration dosage of the drug varies depending on various factors such as progression degree, onset time, age, health condition, complications, etc. of a subject to be administered, but in case of an adult, in one aspect, 1 μg/kg to 200 mg/kg of the composition may be administered in 1 to 3 divided doses per day, and in another aspect, 50 μg/kg to 50 mg/kg may be administered in 1 to 3 divided doses per day.

The pharmaceutical composition may be a formulation for external application to the skin, and the formulation for external application to the skin is a generic term including any formulation applied externally on the skin, and various formulations of medicines may be included therein.

In an exemplary embodiment, the composition comprising a metalloproteinase derived from a strain of the genus Chryseobacterium may comprise 0.0001 to 99.99 wt % of the metalloproteinase derived from a strain of the genus Chryseobacterium based on the total weight of the composition.

In another exemplary embodiment, the composition comprising a metalloproteinase derived from a strain of the genus Chryseobacterium may comprise, based on the total weight of the composition, 0.0001 wt % or more, 0.0005 wt % or more, 0.001 wt % or more, 0.005 wt % or more, 0.01 wt % or more, 0.05 wt % or more, 0.1 wt % or more, or 0.5 wt % or more, and 20 wt % or less, 15 wt % or less, 10 wt % or less, 5 wt % or less, 1 wt % or less, 0.5 wt % or less, 0.1 wt % or less, 0.05 wt % or less, 0.01 wt % or less, 0.005 wt % or less, or 0.001 wt % or less of the metalloproteinase derived from a strain of the genus Chryseobacterium.

For example, the composition comprising a metalloproteinase derived from a strain of the genus Chryseobacterium may comprise, based on the total weight of the composition, 0.0001 to 1 wt %, 0.0001 to 0.5 wt %, 0.0001 to 0.1 wt %, 0.0001 to 0.05 wt %, 0.0001 to 0.01 wt %, 0.0001 to 0.005 wt %, 0.0001 to 0.001 wt %, 0.0005 to 0.001 wt %, or 0.001 to 0.005 wt % of the metalloproteinase derived from a strain of the genus Chryseobacterium.

Hereinafter, the present disclosure will be described in more detail through examples. These examples are only for illustrating the present disclosure, and it will be apparent to those skilled in the art that the scope of the present disclosure is not to be construed as being limited by these examples.

### Example 1. Manufacture of Culture Solution of Strain of the Genus Chryseobacterium

In this Example, a strain of Chryseobacterium camelliae was used as a strain of the genus Chryseobacterium, and the culture solution of the strain of the genus Chryseobacterium was manufactured as follows. As the strain of Chryseobacterium camelliae, a Chryseobacterium camelliae Dolsongi-HT1 strain with a deposit number of KCCM11883P was used. The strain may be obtained according to the isolation and identification method described below, as described in Korean Published Patent Document No. 10-2018-0035682. The document is incorporated herein by reference in its entirety.

(1) Isolation of Microorganism from Green Tea

In order to select a microorganism having keratin degradation activity derived from plants, a liquid minimal medium (M9 medium including 0.5% soluble keratin: CaCl₂) 0.015 g/L, Na₂HPO₄ 6.78 g/L, KH₂PO₄ 3 g/L, NaCl 0.5 g/L, MgSO₄ 0.5 g/L) using keratin as a carbon source and nitrogen source was used to culture the microorganism at 30° C. or 37° C.

The method of selecting the microorganism using the minimal medium is a method of selecting a microorganism of interest according to the growth rate of the microorganism, and microorganisms having low keratinase activity are spontaneously removed at the culturing stage, thereby enabling selection of active microorganisms including enzymes having high activity. The microorganisms capable of growing in the minimal medium means that they have keratinase activity.

Accordingly, the microorganism having keratin degradation activity was isolated from green tea of Dolsongi tea fields, Jeju Island, Korea, in the limited medium.

### (2) Identification of Isolated Microorganism

In order to identify the microorganism having keratinase activity isolated as described above, a DNA sequence encoding a partial sequence of 16S ribosomal RNA of the microorganism was analyzed to identify the species and genus thereof.

After culturing the microorganism in a liquid medium, genomic DNA was extracted from the microorganism, and was amplified by PCR (polymerase chain reaction) method using primers generally used for bacterial identification, 27F (5′-AGAGTTTGATCMTGGCTCAG-3′, SEQ ID NO: 1)/1492R (5′-TACGGYTACCTTGTTACGACTT-3′, SEQ ID NO: 2), and the sequence thereof was analyzed.

As a result of base sequence analysis, it was shown to have 99% homology with a 16S rRNA partial sequence of Chryseobacterium camelliae strain THG C4-1 (KACC 16985; Sequence accession no. (16S rRNA) JX843771), and 98% homology with a 16S rRNA partial sequence of Chryseobacterium taiwanense.

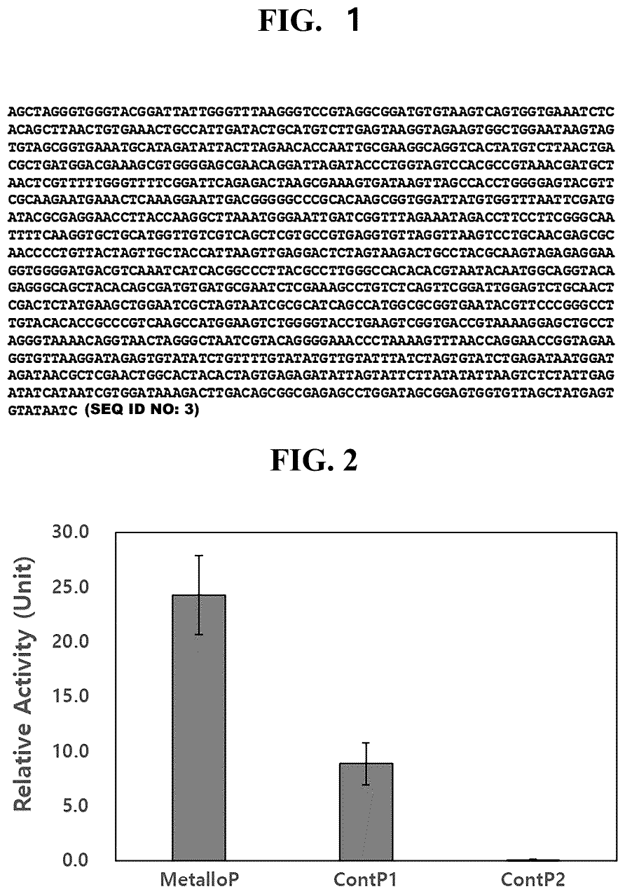

Accordingly, the novel microbial strain isolated and cultured as described above was confirmed to be Chryseobacterium camelliae by molecular phylogenetic analysis based on the 16S rDNA base sequence, and was named Chryseobacterium camelliae Dolsongi-HT1, and the novel microbial strain was deposited under a deposit number of KCCM11883P with the Korean Culture Center of Microorganisms (KCCM), a depository institution, on Sep. 1, 2016. The 16S rDNA partial sequence of Chryseobacterium camelliae Dolsongi-HT1 strain of SEQ ID NO: 3 is illustrated in FIG. 1.

### (3) Manufacture of Culture Solution of Microorganism

The Chryseobacterium camelliae Dolsongi-HT1 strain derived from green tea was seed cultured for 15 hours, and then 10 mL of the cultured strain was inoculated into 1 L of optimized medium (1% Tryptone/1% NaCl/0.5% Skim milk), and cultured at 30° C. at 200 rpm for 15 hours. After culturing, a culture solution from which microorganisms were removed was obtained.

### Example 2. Enzyme Purification and Sequence Analysis from Culture Solution of Strain of the Genus Chryseobacterium

From the culture solution of the strain of the genus Chryseobacterium manufactured in Example 1, proteins having a size of 10 KDa or more were obtained by ultrafiltration using a filter having a cut-off size of 10 KDa. To select proteins having proteolytic activity, proteins were separated with ion exchange resin (Q-SEPHAROSE® HP column, Cytica, Marlborough, MA, USA), and proteins included in active fractions were analyzed with a mass spectrometer (Q EXACTIVE™ Plus mass spectrometer, Thermo Fisher Scientific, Waltham, MA, USA). A total of about 120 proteins were analyzed.

Since the Chryseobacterium camelliae Dolsongi-HT1 strain has completed whole genome analysis, by comparing the whole genome with the protein analysis result, 8 proteins named protease among the analyzed proteins were selected. The selected 8 proteins were made into recombinant proteins as described below, and keratin degradation activities were compared.

First, after amplifying gene sequences of 8 proteins by PCR using forward primers and reverse primers listed in Table 1 below, each enzyme was expressed using Bacillus subtilis secretion protein expression system (TAKARA®). The vector used for manufacture of recombinant proteins was a pBE-S vector, and genes were introduced into the vector using an In-Fusion cloning system. The vector into which the genes were introduced was introduced into host B. subtilis RIK1285. For protein expression, the expression strain was cultured at 37° C. for 24 hours. After culturing, microbial cells were removed, and the culture solution was collected, and then concentrated using ultrafiltration and lyophilized to obtain samples.

TABLE 1

| Protein No | Primer sequence |
|---|---|
| No. 1_Forward | 5′- |
| (SEQ ID NO: 4) | GAAGAAGGAGATATACATATGCAGGACCGCAATGCAGC |
| | ACTT-3′ |
| No. 1_Reverse | 5′- |
| (SEQ ID NO: 5) | GTGGTGGTGGTGGTGGTGCTCGAGCTCTTTAATAACTTT |
| | TTTGGAAA-3′ |
| No. 2_Forward | 5′- |
| (SEQ ID NO: 6) | GCTGCGGCCGGTGCACATATGCAGACTTTTATCCAGGCA |
| | TA-3′ |
| No. 2_Reverse | 5′- |
| (SEQ ID NO: 7) | GTGGTGATGATGGTGATGTCTAGACCTTCCTACCAGGAT |
| | CTTTC-3′ |
| No. 3_Forward | 5′- |
| (SEQ ID NO: 8) | GCTGCGGCCGGTGCACATATGCAACAGCTGAGTGCAGA |
| | ACA-3′ |
| No. 3_Reverse | 5′- |
| (SEQ ID NO: 9) | GTGGTGATGATGGTGATGTCTAGAGTATTTGATAAATTTT |
| | ATAT-3′ |
| No. 4(ContP2)_Forward | 5′- |
| (SEQ ID NO: 10) | GAAGAAGGAGATATACATATGTGCAACACCAATGAAATG |
| | AG-3′ |
| No. 4(ContP2)_Reverse | 5′- |
| (SEQ ID NO: 11) | GTGGTGGTGGTGGTGGTGCTCGAGTGGGGCATCCGGGC |
| | TGATCC-3′ |
| No. 5(MetalloP)_Forward | 5′- |
| (SEQ ID NO: 12) | GAAGAAGGAGATATACATATGAACAATCTGCTACAACAG |
| | CC-3′ |
| No. 5(MetalloP)_Reverse | 5′- |
| (SEQ ID NO: 13) | GTGGTGGTGGTGGTGGTGCTCGAGTTTCAGCTGCTTAA |
| | ATTCTT-3′ |
| No. 6(ContP1)_Forward | 5′- |
| (SEQ ID NO: 14) | GCTGCGGCCGGTGCACATATGGACAACATTGCAAACCA |
| | ACAC-3′ |
| No. 6(ContP1)_Reverse | 5′- |
| (SEQ ID NO: 15) | GTGGTGATGATGGTGATGTCTAGAGTACAATCTCAATCC |
| | TGATCT-3′ |
| No. 7_Forward | 5′- |
| (SEQ ID NO: 16) | GCTGCGGCCGGTGCACATATGCAAAAAAACAACGATGA |
| | AAT-3′ |
| No. 7_Reverse | 5′- |
| (SEQ ID NO: 17) | GTGGTGATGATGGTGATGTCTAGATTCTTTGATCACCTTT |
| | TTGG-3′ |
| No. 8_Forward | 5′- |
| (SEQ ID NO: 18) | GCTGCGGCCGGTGCACATATGCAGCTGGTGTTTGTGTAT |
| | TT-3′ |
| No. 8_Reverse | 5′- |
| (SEQ ID NO: 19) | GTGGTGATGATGGTGATGTCTAGATTCCTTTATGAATTTT |
| | TCAT-3′ |

Keratinase activity was measured using 0.5% (wt/v) keratin azure and pH 8.0 Tris-HCl buffer. When Keratin azure is decomposed by keratinase, azure dye is released, whereby the reaction solution turns blue, so absorbance was measured at 595 nm and the activity was compared (absorbance 0.01=1 Unit).

As a result, keratinase activity was confirmed in two protein samples No. 5 and No. 6, MetalloP and ContP1, and accordingly, MetalloP and ContP1 were confirmed to be capable of providing an effect of skin texture improvement through keratin removal (see FIG. 2). In particular, MetalloP exhibited remarkably high keratinase activity. Meanwhile, protein sample No. 4, ContP2, was the highest in content among the purified proteins, but did not exhibit keratinase activity at all.

As described above, from Chryseobacterium camelliae Dolsongi-HT1 strain isolated from green tea leaves, an enzyme having keratinase activity was purified, and as a result of comparing its sequence with an NCBI database, MetalloP and ContP1 were confirmed to be enzymes belonging to metalloproteinase family. The purified enzyme MetalloP was confirmed to have a sequence of SEQ ID NO: 20. The sequence of MetalloP may be represented by SEQ ID NO: 22 including a signal sequence of SEQ ID NO: 21 suitable for the expression strain, the sequence of ContP1 may be represented by SEQ ID NO: 24 including a signal sequence of SEQ ID NO: 23, and the sequence of ContP2 may be represented by SEQ ID NO: 26 including a signal sequence of SEQ ID NO: 25.

SEQ ID NO: 20
CNDNSTEDNNLLQQPQAESAKVEQPNEREKACYYVDQNWNSSAVLKTTL
KTSTDTNFMNGQMTKIASLWGRNNPTLRFVDDPSSPNSTYNAISYSTGK
IYYGYAIYYDAKNKGGDIVNAMILAHEYGHQLQYIFGLPSVNESTARPN
ELEADGFAGYYLRRPEGYNKTQFSEIAAAYEFAQSIGDYATTSPGHHGT
PPQRRSAVRLGFLLGQYNLSASDFDYNFFYYYQGVLNGTYKMGKNSRNP
ELDAYMMQYIDELRKIQSGEISAEEFKQLK
SEQ ID NO: 21
MKKNFNFCCLAGAIAVFSLTA
SEQ ID NO: 22
MKKNFNFCCLAGAIAVFSLTACNDNSTEDNNLLQQPQAESAKVEQPNER
EKACYYVDQNWNSSAVLKTTLKTSTDTNFMNGQMTKIASLWGRNNPTLR
FVDDPSSPNSTYNAISYSTGKIYYGYAIYYDAKNKGGDIVNAMILAHEY
GHQLQYIFGLPSVNESTARPNELEADGFAGYYLRRPEGYNKTQFSEIAA
AYEFAQSIGDYATTSPGHHGTPPQRRSAVRLGFLLGQYNLSASDFDYNF
FYYYQGVLNGTYKMGKNSRNPELDAYMMQYIDELRKIQSGEISAEEFKQ
LK
SEQ ID NO: 23
MKKLLLGALMLGFMSACNS
SEQ ID NO: 24
MKKLLLGALMLGFMSACNSDNIANQHEQSDNLVSPTGGSALQRGCASEE
VRKIALQNSPELRQRFSALEAQTEKFENDLKLGKVLSDGSVEIPVVVNV
LYRTSSENLSDSRIAEQIAVLNADYAGTNSDVSKIPSEFQSVKSGDVKV
KFRLANTVRKSTTKTSWSTNDAMKRSSSGGIDATSPSNYLNIWVVGNMG
QILGYATFPESAGMWNDGVVIAAPYFGKTGASSPFNLGRTATHEVGHYL
NLRHIWGDANCGNDLVADTPTQTGANSGKPNYPLYNTCSGVQRSVMFMN
YMDYVDDAAMFMFSAGQRTRMQSVVASSGPRSGLRLY
SEQ ID NO: 25
MKTMLKSILTVALLWSAVS
SEQ ID NO: 26
MKTMLKSILTVALLWSAVSCNTNEMSDINSSAQTQTSNSGNFAAKASDN
PENPLNNWEECGKKHNVILTYVITYQKTLDNSGLTNSEIADKCIMASNN
YFNQKYAQDYNNTQSPFTVAKIRSILNDGDNKFSNVIQGMNASDKVKAK
TSELVNMMYNLASTDKNTEYAEVKARIVDFEAGIMNDASLSQAEKDQLL
QTTSIARYSSYLWSNTIDHSDSVSSVTGKKKWWKWVVVAVCDFAGGAAG
TAAGGGVLSVAGAIAGAAGASSGGAALVDWISPDAP

### Example 3. Evaluation of Lipofuscin Degradation Activity of Metalloproteinases Derived from Strain of the Genus Chryseobacterium

Enzymes MetalloP, ContP1, and ContP2, which were overexpressed by preparing recombinant strains in Example 2, were treated to cells in which lipofuscin was induced as described below, and the lipofuscin removal efficacy of the enzymes was evaluated. ContP1 is an enzyme having about 50% keratin degradation efficacy compared to MetalloP, and ContP2 is an enzyme having no keratin degradation efficacy.

Fibroblasts were grown to 90% confluent state, and then 40 μM leupeptin, 45 μM FeCl₃, and 10 μM H₂O₂ were added to the growth medium, and culturing was continued for 10 days so that lipofuscin was accumulated in the cells. The culture solution was replaced every 2 days. After culturing for 10 days, each sample was treated at a concentration of 10 ppm for 7 days. The degree of lipofuscin degradation was measured with a confocal laser scanning microscope (LSM 980, Carl Zeiss, Oberkochen, Germany) equipped with a FITC (fluorescein isothiocyanate) 540/40 filter.

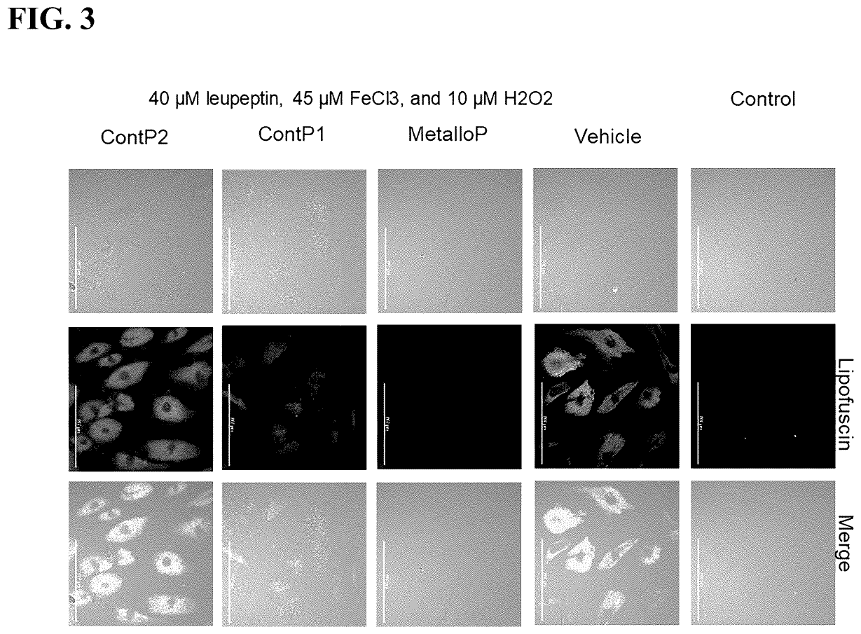

As a result, as illustrated in FIG. 3, in the experimental groups (MetalloP, ContP1) in which metalloproteinases were treated to cells accumulated with lipofuscin, lipofuscin was shown to be removed. In particular, in the experimental group treated with MetalloP, accumulated lipofuscin was completely removed, so it was confirmed that the lipofuscin degradation activity of MetalloP was the most excellent.

Formulation examples of the composition according to one aspect of the present disclosure are described below, but may also be applied in various other formulations, and this is not intended to limit the present disclosure, but only to describe it in detail.

### Formulation Example 1. Softening Cosmetic Water

0.01 wt % of metalloproteinase MetalloP obtained in Example 2, 3 wt % of glycerin, 2 wt % of butylene glycol, 2 wt % of propylene glycol, 0.1 wt % of carboxyvinyl polymer, 10 wt % of ethanol, 0.1 wt % of triethanolamine, a small amount of preservative, a small amount of pigment, a small amount of fragrance, and balance of purified water were mixed, and softening cosmetic water was manufactured according to a conventional manufacturing method.

### Formulation Example 2. Nourishing Cosmetic Water

0.01 wt % of metalloproteinase MetalloP obtained in Example 2, 4 wt % of beeswax, 1.5 wt % of polysorbate 60, 0.5 wt % of sorbitan sesquioleate, 5 wt % of liquid paraffin, 5 wt % of squalane, 5 wt % of caprylic/capric triglyceride, 3 wt % of glycerin, 3 wt % of butylene glycol, 3 wt % of propylene glycol, 0.1 wt % of carboxyvinyl polymer, 0.2 wt % of triethanolamine, a small amount of preservative, a small amount of pigment, a small amount of fragrance, and balance of purified water were mixed, and nourishing cosmetic water was manufactured according to a conventional manufacturing method.

### Formulation Example 3. Nourishing Cream

0.01 wt % of metalloproteinase MetalloP obtained in Example 2, 10 wt % of beeswax, 1.5 wt % of polysorbate 60, 0.5 wt % of sorbitan sesquioleate, 10 wt % of liquid paraffin, 5 wt % of squalane, 5 wt % of caprylic/capric triglyceride, 5 wt % of glycerin, 3 wt % of butylene glycol, 3 wt % of propylene glycol, 0.2 wt % of triethanolamine, a small amount of preservative, a small amount of pigment, a small amount of fragrance, and balance of purified water were mixed, and nourishing cream was manufactured according to a conventional manufacturing method.

### Formulation Example 4. Pack

0.01 wt % of metalloproteinase MetalloP obtained in Example 2, 13 wt % of polyvinyl alcohol, 0.2 wt % of sodium carboxymethyl cellulose, 0.1 wt % of allantoin, 5 wt % of ethanol, 0.3 wt % of nonylphenyl ether, a small amount of preservative, a small amount of pigment, a small amount of fragrance, and balance of purified water were mixed, and a pack was manufactured according to a conventional manufacturing method.

### Formulation Example 5. Pharmaceutical Formulation for Topical Administration (Patch)

According to the composition described in Table 2 below, a pharmaceutical formulation for topical administration (patch) was manufactured by a conventional method.

TABLE 2

| Raw material | Content (wt %) |
|---|---|
| Metalloproteinase MetalloP obtained in Example 2 | 2.0 |
| Beta-1,3-glucan | 3.0 |
| Diethylamine | 0.7 |
| Sodium sulfite | 0.1 |
| Polyoxyethylene lauryl ether (E.O = 9) | 1.0 |
| Polyhydroxyethylene cetyl stearyl ether | 1.0 |
| (Cetomacrogol 1000) | |
| Viscous paraffin oil | 2.5 |
| Caprylic acid ester/Capric acid ester (Cetiol LC) | 2.5 |
| Polyethylene glycol 400 | 3.0 |
| Polyacrylic acid (Carbopol 934P) | 1.0 |
| Purified water | balance |
| total | 100 |

### Formulation Example 6. Unit-Dose Powder

2 g of metalloproteinase MetalloP obtained in Example 2 and 1 g of lactose were mixed, and after filling into an airtight pouch, a unit-dose powder was manufactured.

### Formulation Example 7. Tablet

100 mg of metalloproteinase MetalloP obtained in Example 2, 100 mg of corn starch, 100 mg of lactose, and 2 mg of magnesium stearate were mixed, and after tableting according to a conventional manufacturing method, a tablet was manufactured.

### Formulation Example 8. Capsule

100 mg of metalloproteinase MetalloP obtained in Example 2, 100 mg of corn starch, 100 mg of lactose, and 2 mg of magnesium stearate were mixed, and after filling into a gelatin capsule according to a conventional manufacturing method, a capsule was manufactured.

### Formulation Example 9. Pill

1 g of metalloproteinase MetalloP obtained in Example 2, 1.5 g of lactose, 1 g of glycerin, and 0.5 g of xylitol were mixed, and according to a conventional method, a pill was manufactured to contain 4 g per pill.

### Formulation Example 10. Granule

150 g of metalloproteinase MetalloP obtained in Example 2, 50 mg of soybean extract, 200 mg of glucose, and 600 mg of starch were mixed, 100 mg of 30% ethanol was added, and after drying at 60° C., granules were formed and filled into a pouch, thereby a granule was manufactured.

### Formulation Example 11. Drink

50 mg of metalloproteinase MetalloP obtained in Example 2, 10 g of glucose, 0.6 g of citric acid, and 25 g of liquid oligosaccharide were mixed, 300 ml of purified water was added, and 200 ml each was filled into bottles. After filling into bottles, sterilization was performed at 130° C. for 4 to 5 seconds, and a drink was manufactured.

### Formulation Example 12. Caramel

50 mg of metalloproteinase MetalloP obtained in Example 2, 1.8 g of corn syrup, 0.5 g of skim milk, 0.5 g of soybean lecithin, 0.6 g of butter, 0.4 g of hydrogenated vegetable oil, 1.4 g of sugar, 0.58 g of margarine, and 20 mg of sodium chloride were mixed and molded, and a caramel was manufactured.

The above describes specific aspects of the present disclosure in detail. It will be apparent to those skilled in the art that these specific techniques are merely preferred embodiments and that the scope of the present disclosure is not limited thereby. Therefore, the substantial scope of the present disclosure is defined by the appended claims and their equivalents.
Deposit Number
Depository institution: Korean Culture Center of Microorganisms (KCCM)
Deposit Number: KCCM11883P
Date of deposition: 20160901

What is claimed is:

1. A method for skin anti-aging, skin lightening, or skin texture improvement, comprising:
applying to a subject in need thereof a composition comprising an effective amount of a metalloproteinase derived from a strain of the genus Chryseobacterium.

2. The method of claim 1, wherein the strain is a strain of Chryseobacterium camelliae.

3. The method of claim 1, wherein the strain is a Chryseobacterium camelliae Dolsongi-HT1 strain with a deposit number of KCCM11883P.

4. The method of claim 1, wherein the metalloproteinase comprises an amino acid sequence of SEQ ID NO: 20 or SEQ ID NO: 22.

5. The method of claim 1, wherein the skin anti-aging, the skin lightening, or the skin texture improvement is obtained by inhibition of lipofuscin accumulation or by removal of lipofuscin.

6. A method for inhibiting lipofuscin accumulation or for removing lipofuscin, comprising:
applying to a subject in need thereof a composition comprising an effective amount of a metalloproteinase derived from a strain of the genus Chryseobacterium.

7. The method of claim 6, wherein the strain is a strain of Chryseobacterium camelliae.

8. The method of claim 6, wherein the strain is a Chryseobacterium camelliae Dolsongi-HT1 strain with a deposit number of KCCM11883P.

9. The method of claim 6, wherein the metalloproteinase comprises an amino acid sequence of SEQ ID NO: 20 or SEQ ID NO: 22.

10. The method of claim 6, wherein the method is for preventing, improving, or treating a disease caused by lipofuscin accumulation.

11. The method of claim 10, wherein the disease caused by lipofuscin accumulation comprises one or more selected from the group consisting of sarcopenia, progeria, Alzheimer's disease, Parkinson's disease, amyotrophic lateral sclerosis (ALS), myocardial infarction, age-related macular degeneration, neuronal ceroid lipofuscinoses (NCL), acromegaly, denervation atrophy, and chronic obstructive pulmonary disease (COPD).
